(19) United States
(12) Patent Application Publication
(10) Pub. No.: US 2011/0071540 A1
(43) Pub. Date: Mar. 24, 2011

(54) LEAD WITH DISTAL ENGAGEMENT FEATURE TO FACILITATE LEAD PLACEMENT

(75) Inventors: John E. Kast, Hugo, MN (US); James A. Zimmerman, Blaine, MN (US); Craig S. Pilarski, Ham Lake, MN (US); William C. Phillips, Brooklyn Park, MN (US); Thomas I. Miller, Blaine, MN (US); Mary L. Boatwright, Andover, MN (US)

(73) Assignee: Medtronic, Inc

(21) Appl. No.: 12/992,954

(22) PCT Filed: May 29, 2009
(86) PCT No.: PCT/US09/45576

§ 371 Date: Nov. 16, 2010

Related U.S. Application Data
(60) Provisional application No. 61/058,287, filed on Jun. 3, 2008.

(51) Int. Cl. A61B 19/00

(52) U.S. Cl. 606/129

(57) ABSTRACT

An implantable medical lead includes a proximal portion including a contact. The lead also includes a distal portion having a paddle-shaped portion, an electrode, and an engagement element configured to cooperate with a lead advancement tool to facilitate placement of the lead such that distal advancement of the tool relative to the lead pushes the lead distally. The electrode is electrically coupled to the contact, and the engagement element is distal to the electrode. The engagement element is integrally formed with the paddle-shaped portion.

### FIELD

The present disclosure relates to implantable medical devices; more particularly to medical leads having a distal engagement element to facilitate placement of the lead during implantation.

### BACKGROUND

Headaches, such as migraines, and occipital neuralgia are often incapacitating and may lead to significant consumption of drugs to treat the symptoms. However, a rather large number of people are unresponsive to drug treatment, leaving them to wait out the episode or to resort to coping mechanisms. For refractive occipital neuralgia, nerve ablation or separation may effectively treat the pain.

Occipital nerve stimulation may serve as an alternative for treatment of migraines or occipital neuralgia. For example, a dual channel implantable electrical generator may be implanted subcutaneously in a patient. A distal portion of first and second leads may be implanted in proximity to a left and right occipital nerve such that one or more electrode of the leads are in electrical communication with the occipital nerves. The proximal portions of the leads may then be connected to the signal generator such that electrical signals can be delivered from the signal generator to the electrodes to apply therapeutic signals to the occipital nerves. Alternatively, two single channel implantable electrical generators may be employed, where the first lead is connected to one signal generator and the second lead is connected to the second signal generator. In either case, the lead is typically tunneled subcutaneously from site of implantation of the signal generator to the occipital nerve or around the base of the skull. Such tunneling can be time consuming and is invasive.

Implanting the distal portions of the leads in proximity to a left and right occipital nerve such that one or more electrode of the leads are in electrical communication with the occipital nerves can be challenging or invasive, particularly with surgical leads or leads having paddle-shaped distal portions. Typically, an incision is made in the skin of the patient to allow for implantation of the distal portions of such leads. Because of the size and shape of the distal portions of paddle leads, they cannot be implanted using typical percutaneous techniques. It would be desirable to implant paddle or surgical leads in a patient in a less invasive manner.

### BRIEF SUMMARY

The present disclosure describes, among other things, leads having an engagement element configured to cooperate with an engagement tool such that distal advancement of the engagement tool relative to the lead pushes the lead when the tool is engaged with the engagement element. Such engagement features may be particularly desirable for surgical or paddle leads having distal end portions that may be pushed through tissue of a patient for short distances.

In an embodiment, a method for pushing a distal portion of a lead through tissue of a patient is described. The distal portion of the lead has an electrode and an engagement element distal the electrode. The method includes engaging the engagement element of the lead with an engagement tool, and advancing the tool distally relative to the lead to push the distal portion of the lead through the tissue. The lead may be pushed by distal advancement of the tool until the electrode is positioned in a desired location of the tissue.

In an embodiment, a system for implanting a lead is described. The system includes a lead having a distal portion that includes an electrode and an engagement element distal the electrode. The system also includes an engagement tool having a lead engagement feature and an elongate member extending from the lead engagement feature such that distal advancement of the elongate member, when the lead engagement feature is engaged with the engagement element of the lead, pushes the lead distally.

In an embodiment, an implantable medical lead is described. The lead includes a proximal portion including a contact. The lead also includes a distal portion having a paddle-shaped portion, an electrode, and an engagement element configured to cooperate with a lead advancement tool to facilitate placement of the lead such that distal advancement of the tool relative to the lead pushes the lead distally. The electrode is electrically coupled to the contact, and the engagement element is distal to the electrode. The engagement element is integrally formed with the paddle-shaped portion.

In an embodiment, a method for applying electrical signals to left and right occipital nerves of a patient is described. The method includes implanting a lead including a proximal portion, a first distal arm, a second distal arm, and a branch region between the proximal portion and the first and second distal arms. The proximal portion includes first and second contacts. The first distal arm includes an electrode electrically coupled to the first contact and has an engagement element distal to the electrode. The second distal arm includes an electrode electrically coupled to the second contact and has an engagement element distal to the electrode. Implanting the lead includes engaging the engagement element of the first distal arm with a first engagement tool and advancing the tool distally relative to the lead to push the distal arm of the lead through tissue of the patient until the electrode is positioned adjacent to the left occipital nerve. Implanting the lead further includes engaging the engagement element of the second distal arm with a second engagement tool and advancing the tool distally relative to the lead to push the distal arm of the lead through tissue of the patient until the electrode is positioned adjacent to the right occipital nerve. The first and second engagement tools may be the same or may be different. The method further includes (i) operably coupling the first and second contacts of the proximal portion of the lead with an implantable signal generator; (ii) delivering a first signal generated by the signal generator to the left occipital nerve via the electrode of the first distal arm of the lead, and (iii) delivering a second signal generated by the signal generator to the right occipital nerve via the electrode of the second distal arm of the lead. The first and second signal may be the same or different. In some embodiments, a signal is delivered between the first and second electrodes to apply the signal to the left or right occipital nerve.

The leads, extensions, signal generators, systems and methods described herein provide one or more advantages over prior leads, extensions, signal generators, systems and methods. Such advantages will be readily understood from the following detailed description when read in conjunction with the accompanying drawings.

### BRIEF DESCRIPTION OF THE DRAWINGS

FIG. 1 is a schematic side view of an implantable system including a signal generator, lead extension and lead.

FIGS. 2A, 3, and 4A are schematic top-down views of representative leads or distal portions of leads having an engagement element.

FIGS. 2B and 4B are schematic bottom-up views of embodiments of (or alternatives of) distal portions of leads shown in FIGS. 2A and 2B, respectively.

FIGS. 5 and 6A are schematic side views of distal portions of leads having an engagement element.

FIG. 6B is a schematic perspective view of an embodiment of the distal portion of the lead shown in FIG. 6A.

FIGS. 7-9 are schematic side views of embodiments of engagement tools.

FIGS. 10A-C, 11A-B, 12A-D, and 13A-D are schematic views of engagement tools pushing leads via interaction with an engagement element.

FIGS. 14A-B are schematic diagrams showing distal portions of bifurcated leads implanted in a subjects and positioned to apply an electrical signal to left and right occipital nerves.

FIG. 15A is a schematic side view of a representative bifurcated lead.

FIGS. 15B-D are schematic cross-sections of alternative embodiments of the proximal portion of the lead shown in FIG. 15A taken through line 15b-15b.

FIG. 15E is a schematic side view of an embodiment of the branch region of the lead depicted in FIG. 15A, showing conductors running through the branch region.

FIGS. 16-17 are schematic side views of representative bifurcated leads.

FIGS. 18-19 are schematic side views of lead extensions having a connector configured to operably couple to leads and associated leads.

FIG. 20 is a schematic cross-section of a connector having receptacles for receiving leads.

FIGS. 21A-E are schematic side views of representative bifurcated leads having extensible portions.

FIGS. 22A-F are schematic side views of representative bifurcated leads having attached anchors.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”.

“Exemplary” or “representative” is used herein in the sense of “for example” or “for the purpose of illustration”, and not in a limiting sense.

The present disclosure describes, among other things, leads having an engagement element configured to cooperate with an engagement tool such that distal advancement of the engagement tool relative to the lead pushes the lead when the tool is engaged with the engagement element. Such engagement features may be particularly desirable for surgical or paddle leads having distal end portions that may be pushed through tissue of a patient for short distances.

Nearly any implantable medical device or system employing leads may be used in conjunction with the leads described herein. Representative examples of such implantable medical devices include hearing implants, cochlear implants; sensing or monitoring devices; signal generators such as cardiac pacemakers or defibrillators, neurostimulators (such as spinal cord stimulators, brain or deep brain stimulators, peripheral nerve stimulators, vagal nerve stimulators, occipital nerve stimulators, subcutaneous stimulators, etc.), gastric stimulators; or the like. For purposes of occipital nerve stimulation, electrical signal generators such as Medtronic, Inc.'s Restore® or Synergy® series of implantable neurostimulators may be employed.

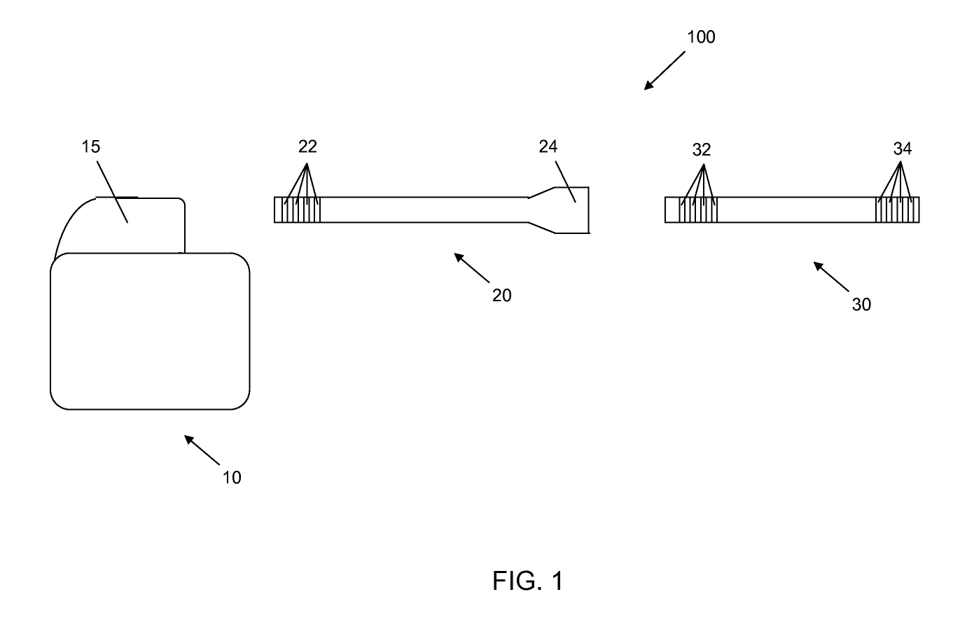

Referring to FIG. 1, a schematic side view of a representative electrical signal generator system 100 is shown. In the depicted system 100, the electrical signal generator 10 includes a connector header 15 configured to receive a proximal portion of lead extension 20. The proximal portion of lead extension 20 contains a plurality of electrical contacts 22 that are electrically coupled to internal contacts (not shown) at distal connector 24 of lead extension 20. The connector header 15 of the signal generator 10 contains internal contacts (not shown) and is configured to receive the proximal portion of the lead extension 20 such that the internal contacts of the connector header 15 may be electrically coupled to the contacts 22 of the lead extension 20 when the lead extension 20 in inserted into the header 15.

The system depicted in FIG. 1 further includes a lead 30. The depicted lead 30 has a proximal portion that includes a plurality of contacts 32 and a distal portion that includes a plurality of electrodes 34. Each of the electrodes 34 may be electrically coupled to a discrete contact 32. The distal connector 24 of the lead extension 20 is configured to receive the proximal portion of the lead 30 such that the contacts 32 of the lead 30 may be electrically coupled to the internal contacts of the connector 24 of the extension 20. Accordingly, a signal generated by the signal generator 10 may be transmitted to a patient by an electrode 34 of lead 30 when lead is connected to extension 20 and extension 20 is connected to signal generator 10.

It will be understood that lead 30 may be coupled to signal generator 10 without use of an extension 20. Any number of leads 30 or extensions 20 may be coupled to signal generator 10. Typically, one or two leads 30 or extensions 20 are coupled to signal generator 10. While lead 20 is depicted as having four electrodes 34, it will be understood that lead 30 may include any number of electrodes 34, e.g. one, two, three, four, five, six, seven, eight, sixteen, thirty-two, or sixty-four. Corresponding changes in the number of contacts 32 in lead 30, contacts 22 and internal contacts in connector 24 of lead extension, or internal contacts in connector 15 of signal generator 10 may be required or desired.

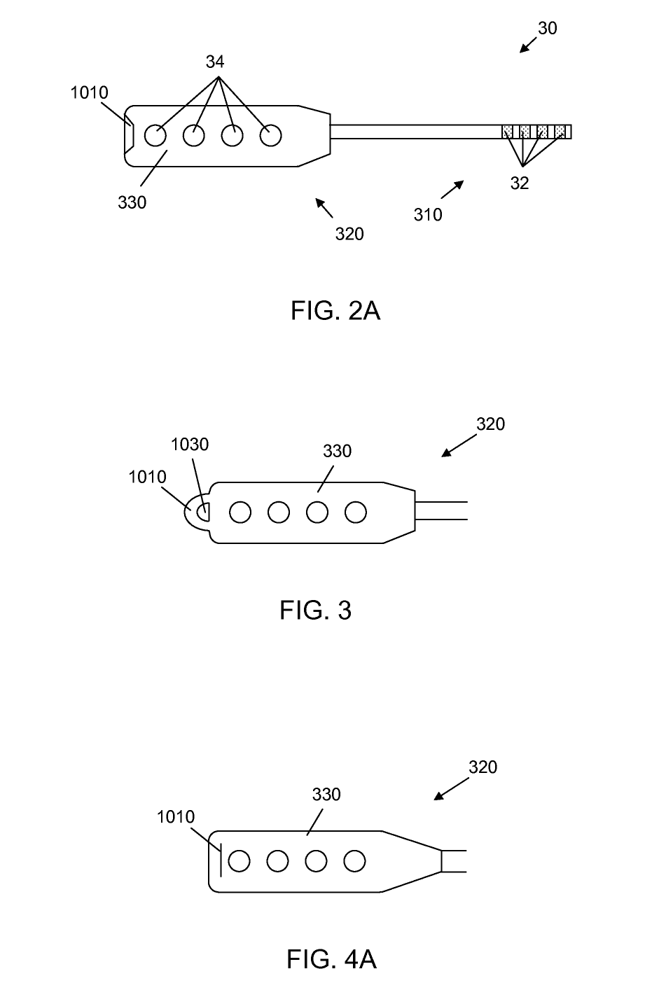

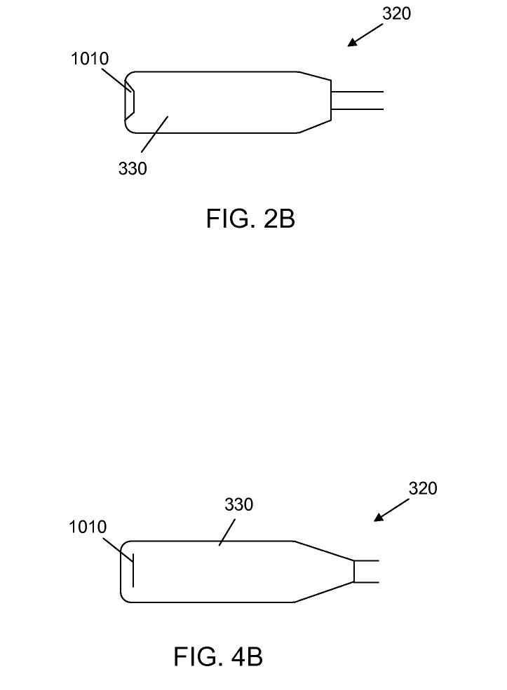

Referring now to FIGS. 2-6, various schematic views of leads 30 or distal portions 320 thereof, having engagement elements 1010 are shown. As shown in FIG. 2A, the leads 30 include proximal portions 310 having one or more contacts 32 and distal portions 320 having one or more electrodes 34 operably coupled to the contacts 32, e.g. as described above. As further shown in FIG. 2A, the depicted leads 320 include paddle-shaped portions 330. The paddle shaped portion 330 includes the one or more electrodes 34 and the engagement element 1010. The engagement element 1010 is distal to the distal most electrode. The engagement element 1010 may be integrally formed with the paddle-shaped portion 330 or attached to the paddle-shaped portion (e.g., adhered, fastened, integrally formed, or otherwise secured).

With reference to FIGS. 2A, 3, and 4A, schematic top-down views of representative leads 30 or distal portions 320 of leads having a variety of engagement element 1010 configurations are shown. As depicted in FIG. 3, the engagement element 1010 may form a hole that may be engaged by a lead advancement tool, such as a tool may have, for example, a hook. In the embodiment depicted in FIG. 4A, the engagement element 1010 includes or consists of a slit in the paddle-shaped portion 330 of the lead. A lead advancement tool may be inserted into the body of the paddle 330 to push the paddle to a desired implant location. In the embodiments depicted in FIGS. 2A and 4A, the engagement element 1010 extends from or is on the surface of the paddle 330 through which the electrodes are exposed. Typically paddle-shaped leads have electrodes exposed through one surface of the paddle, but not through the opposing surface. As shown in the embodiments depicted in FIGS. 2B and 4B, an engagement element 1010 may alternatively or additionally extends from, or may be on, the opposing surface of the paddle 330 through which the electrodes are not exposed.

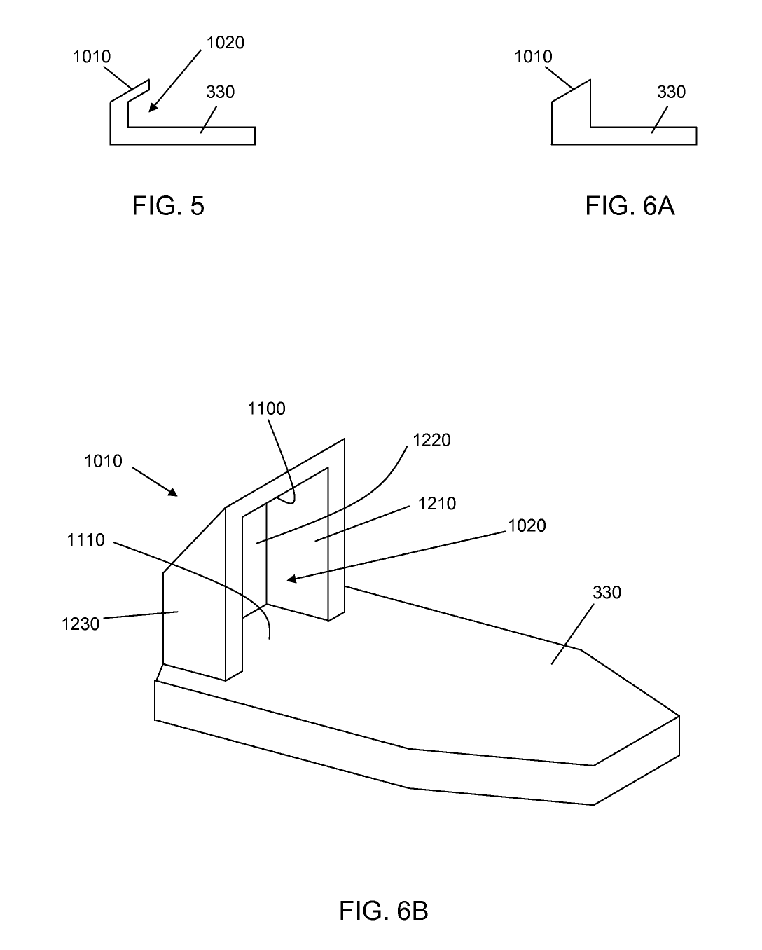

Referring now to FIGS. 5 and 6A, schematic side views of alternative embodiments the distal portion of the lead depicted in FIG. 2B are shown. The engagement element 1010 extends from a major surface of the paddle 330. As depicted in FIG. 5, the engagement element 1010 forms a cavity 1020 configured to receive an engagement tool.

Referring to FIG. 6B, a schematic perspective view of an embodiment of the paddle-shaped portion 330 of the lead depicted in FIG. 6A is shown. As with the engagement element depicted in FIG. 5, the engagement element 1010 depicted in FIG. 6A forms a cavity configured to receive an engagement tool. The cavity 1020 depicted in FIG. 6B is formed by first 1210, second 1220, and third 1230 side walls, a floor 1110, which may be even with the major surface of the paddle 330 or may be recessed relative to the major surface, and a ceiling 1100. The cavity 1020 depicted in FIG. 6B, or other similar cavities, allow the portion of an engagement tool received by the cavity 1020 to engage a variety of surfaces 1100, 1110, 1210, 1220, 1230 to allow for steering or guiding of the distal portion of the lead as it is pushed through tissue of a patient by the tool.

It will be understood that the engagement elements 1010 depicted in FIGS. 2-6 are merely examples of engagement elements that may be employed in accordance with the teaching presented herein. Any other engagement element having a suitable configuration for engaging a portion of an engagement tool such that, when engaged by the tool, distal advancement of the tool pushes the distal portion of the lead distally.

It will be further understood that a lead engagement element may be positioned at any suitable location of the distal portion of the lead. Placing the engagement element distal to the distal most electrode or at or near the distal end of the lead allows for the remainder of the lead to be pulled through the patient's tissue by the pushing force applied to the distally located engagement element. However, if the lead is suitable designed (e.g., sufficiently rigid) to be pushed from a more proximal location, the engagement element may be place in a location more proximal than at or near the distal end of the lead. It will also be understood that an engagement element may be incorporated at any suitable position of a lead, such as the side or mid-body of the paddle portion. It will be further understood that the percutaneous leads, having generally cylindrical distal portions, or leads other that surgical or paddle leads may include engagement elements and may be implanted as described herein.

Engagement elements may be formed of any suitable material. In various embodiments, an engagement element is formed of material that forms the body of the paddle, such as polymeric material. Reinforcing elements may be included in the engagement members to provide sufficient structural rigidity to allow the lead to be pushed through tissue of the patient.

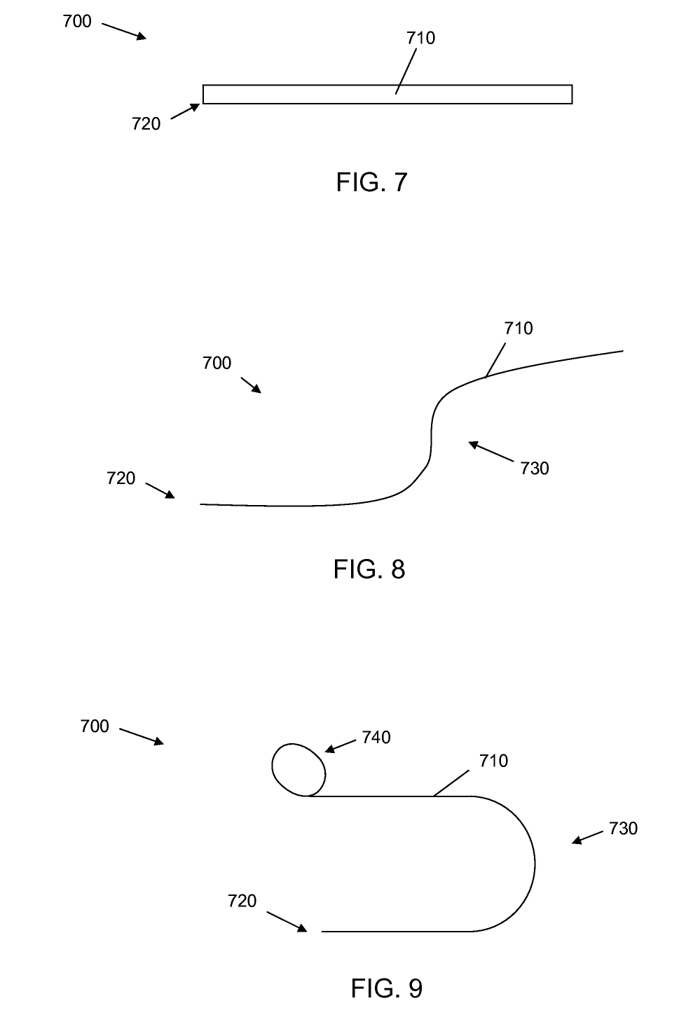

Referring now to FIGS. 7-9, schematic side views of alternative embodiments of engagement tools 700 are shown. The tools 700 have a lead engagement feature 720 configured to engage an engagement element of a lead. The tools 700 also include elongate members 710 that extend proximally from the lead engagement feature 720. In various embodiments, the lead engagement feature 720 is the distal end of the elongate member 710. As shown in FIGS. 8-9, the elongate members may include a curved portion 730. In some embodiments, the tools 700 are preformed to include the curved portion 730. In some embodiments, the elongate members 710 are configured to be manually bent to include a curve portion 730, as needed or desired, by a physician or other health care provider during the implant procedure. The tool 700 depicted in FIG. 9 is bent in a manner such that pulling on a portion, such as the loop 740, of the elongate member 710 distal to the engagement feature 720 cause a portion of the elongate member 710 proximal to the engagement feature 720 to push the engagement feature.

It will be understood that FIGS. 7-9 depict only some examples of suitable configurations for engagement tools that may be employed as described herein. Any other suitable form or configuration of engagement tool may be employed.

An engagement tool may be formed from any suitable material, such as a rigid polymeric material, a metallic material, combinations thereof, or the like. Preferably, the engagement tool is formed of material sufficiently stiff to push a lead through subcutaneous tissue of a patient, yet flexible enough to bend as may be needed during implantation.

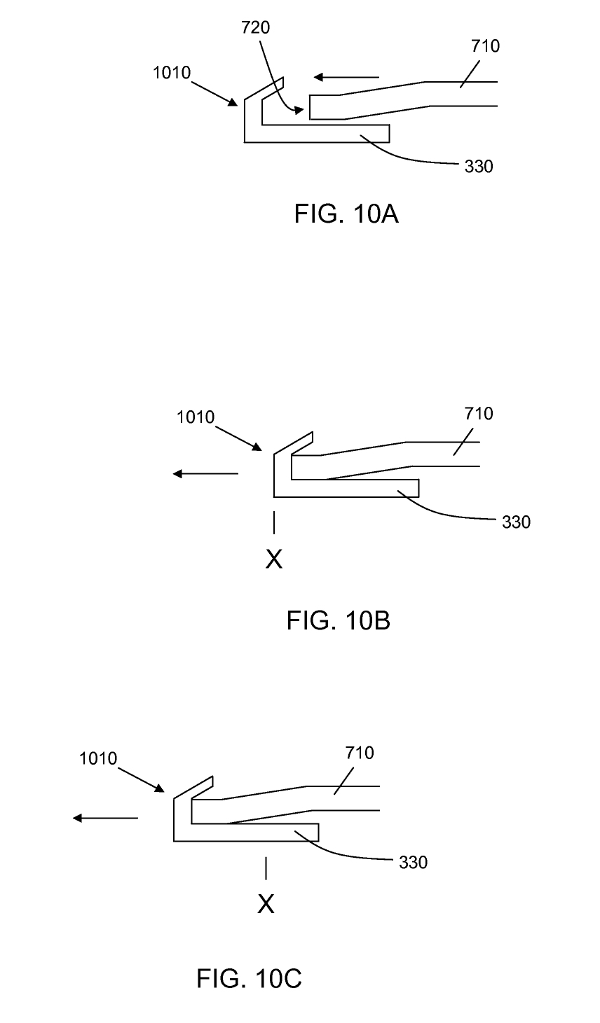

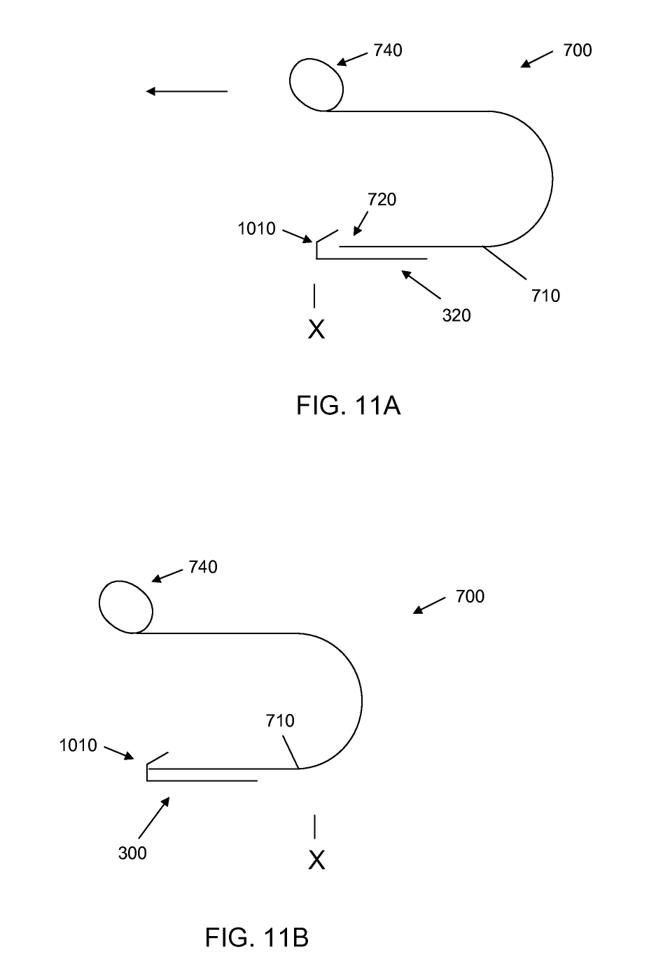

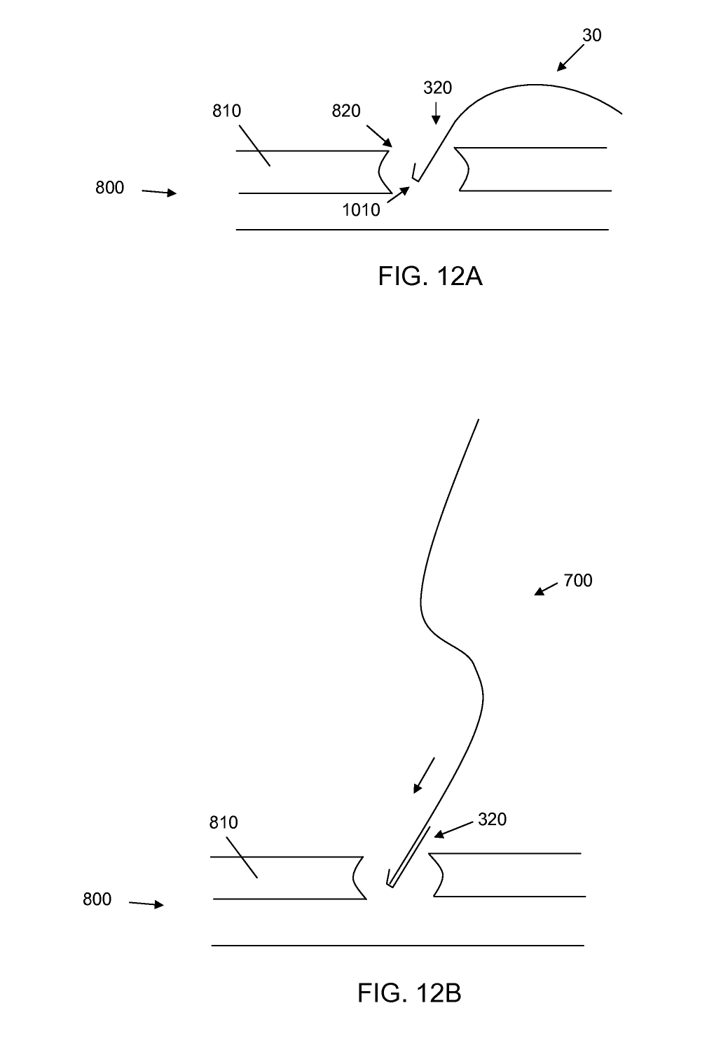

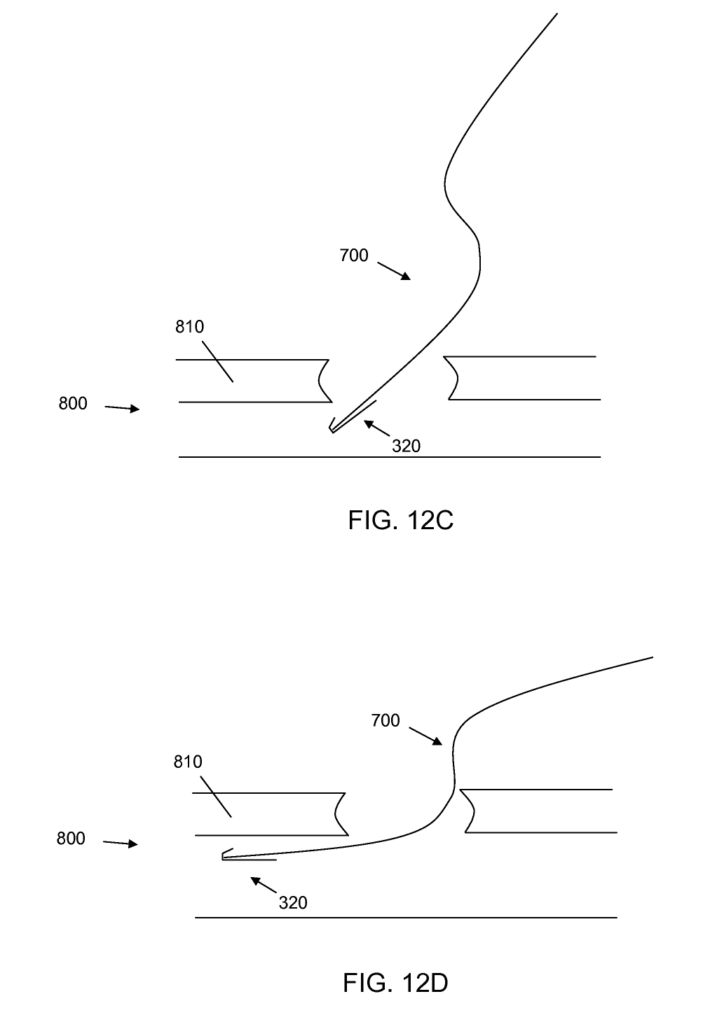

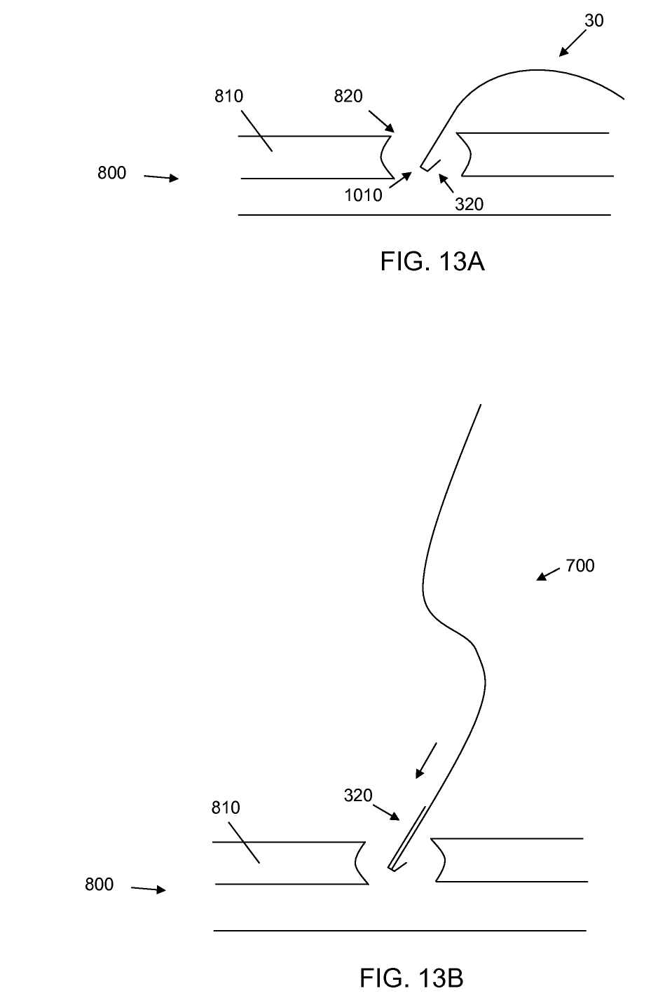

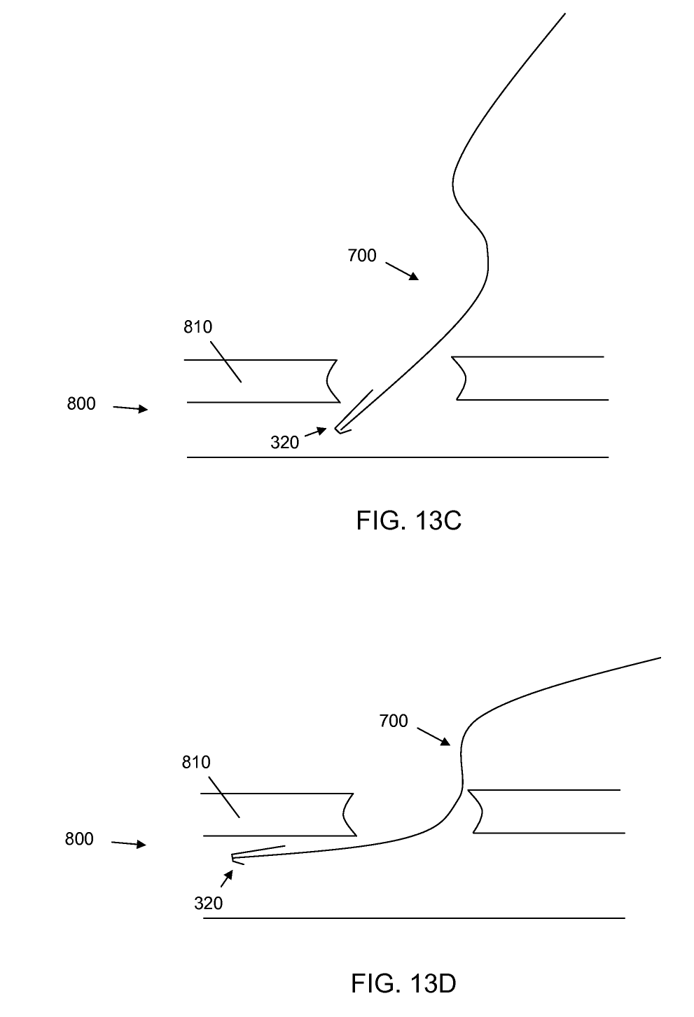

Referring now to FIGS. 10A-C, side views illustrating a tool pushing a distal portion of a lead (only distal portion shown for purposes of brevity, simplicity, and clarity). As shown in FIG. 10A-B, the elongate member 710 in proximity to the engagement feature 720 of a tool may be advanced distally relative to the lead until the engagement feature engages the engagement member 1010 of the paddle-shaped portion 330 of the lead. As shown in FIGS. 10B-C, further distal advancement of the elongate member 710 relative to the lead, when the tool is engaged with the engagement element 1010, causes the distal portion of the lead (including the paddle 330 in the depicted embodiment) to move distally. Position “X” indicated in FIGS. 10B-C is intended to mark a stationary position to reflect movement of the paddle portion 330 of the lead, and the elongate member 710 is pushed against the engagement feature 1010.

FIGS. 11A-B illustrate another example of a tool 700 moving a lead (only the distal portion 320 is shown for the purposes of brevity, simplicity, and clarity). The elongate member 710 distal to the engagement element 720 is pulled, e.g. by pulling on loop 740, to cause the elongate member 710 in proximity to the engagement feature 720 of the tool 700 to push the engagement feature 720. When the engagement feature 720 engages the engagement element 1010 at the distal portion 320 of the lead, distal advancement of the tool, causes the distal portion 320 of the lead to be moved distally.

Referring now to FIGS. 12A-D and FIGS. 13A-D, schematic drawings illustrating the advancement of a distal portion 320 of a lead 30 through tissue of a subject are shown. FIGS. 13A-D are substantially the same as FIGS. 12A-D, except that the orientation of the lead 30 is slightly different. It will be understood that only the distal portion 320 of the lead is shown in FIGS. 12B-D and FIGS. 13B-D for purposes of brevity, simplicity and clarity. As in FIGS. 10-11, the distal portion 320 of the lead includes and engagement element 1010 configured to cooperate with a tool 700 to advance the distal portion 320 of the lead through tissue 800 of a patient. The distal portion 320 of the lead 30 may be inserted through an incision 820 made in the patient. In the depicted embodiment, the incision 820 is through the skin 810 allowing advancement and implantation of the lead 30 in subcutaneous tissue 800 of the patient. A tool 700 (e.g. as described above) may be used to facilitate initial insertion into the subcutaneous tissue 800 (see, e.g., FIG. 12B, 13B) and is used to advance the distal portion 320 of the lead through the tissue 300 (see, e.g., FIGS. 12C-D, 13C-D). As the distal portion 320 of the lead enters the tissue 800 and is pushed through the tissue 800, the angle of the tool 700 (compare FIGS. 12B-D, 13B-D) is manipulated to implant the distal portion 320 of the lead at the appropriate angle and depth within the tissue 800. In the depicted embodiment, the tool 700 is pre-bent or curved. However, in various embodiments, the tool 700 may be bent or curved manually as needed or desired. Once the distal portion 320 of the lead is advanced to the desired location within the tissue 800, the tool 700 may be removed.

In some embodiments, the tool may be removed simply by withdrawing the tool from the tissue. However, in some embodiments, the engagement element of the lead and the engagement feature of the tool may be configured such that a significant amount of force is needed to disengage the tool from the engagement element of the lead (e.g., a compression fit, interference fit, snap fit, or the like). In such embodiments, it may be necessary to employ another tool to hold the distal portion on the lead in place while the engagement tool is disengaged to prevent movement of the distal portion of the lead from its desired implant location. Any suitable additional tool, such as forceps, pliers or the like to hold the paddle portion or the like, may be employed. Alternatively or in addition, the tool may have a mechanical disengaging mechanism to release the lead.

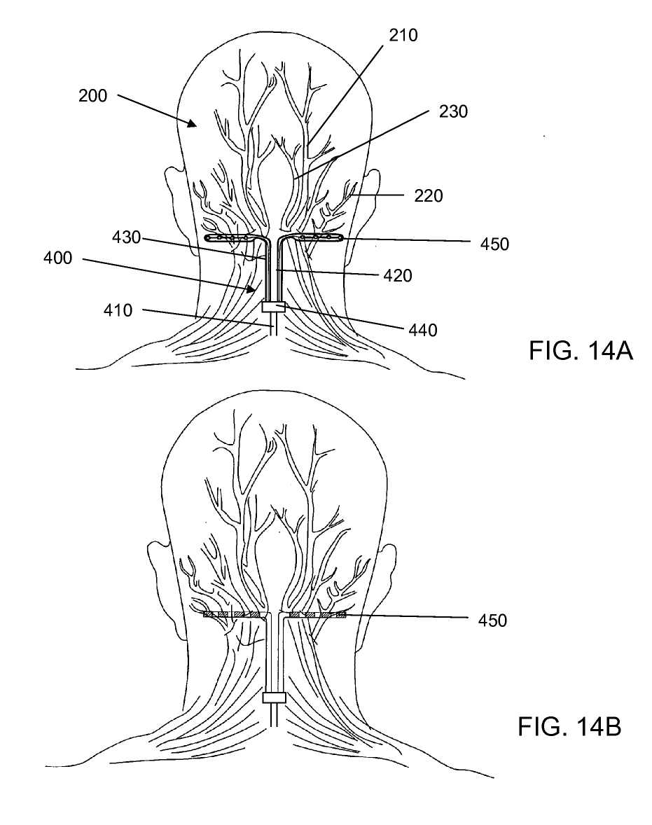

Referring now to FIGS. 14A-B, a bifurcated lead 400 is shown implanted in a patient to provide bilateral therapy to left and right occipital nerves 200. As used herein, occipital nerve 200 includes the greater occipital nerve 210, the lesser occipital nerve 220 and the third occipital nerve 230. The greater and lesser occipital nerves are spinal nerves arising between the second and third cervical vertebrae (not shown). The third occipital nerve arises between the third and fourth cervical vertebrae. The portion of the occipital nerve 200 to which an electrical signal is to be applied may vary depending on the disease to be treated and associated symptoms or the stimulation parameters to be applied. In various embodiments, the lead distal portions 450 that contain electrodes are placed to allow bilateral application of electrical signals to the occipital nerve 200 at a level of about C1 to about C2 or at a level in proximity to the base of the skull. The position of the electrode(s) may vary. It will be understood that the electrode need not, and in various embodiments preferably does not, contact the nerve to apply the signal to the nerve. It will be further understood that a signal may be applied to any suitable portion of an occipital nerve, whether at a trunk, branch, or the like. In various embodiments, one or more electrodes are placed between about 1 cm and about 8 cm from the midline to effectively provide an electrical signal to the occipital nerve 200.

As shown in FIG. 14A, a bifurcated lead 400 may include a paddle shaped distal portion 450 containing electrodes. Such paddle shaped leads are often referred to as surgical leads. Examples of surgical leads that may be modified to form paddle leads as described herein include Medtronic Inc.'s Resume, SymMix, On-Point, or Specify series of leads. Surgical leads typically contain electrodes that are exposed through one face of the paddle, providing directional stimulation. The depicted bifurcated lead 200 also includes a single proximal portion 410 that allows for only one tunneling procedure to the signal generator (not shown) implant site. In addition, the bifurcated lead 400 contains a branch region 440 and first 420 and second 430 distal arms. As shown in FIG. 14B, the bifurcated lead may include distal portion 450 that include electrodes that are generally cylindrically shaped. Such leads are often referred to percutaneous leads. Examples of percutaneous leads that may be modified to form leads as described herein include Medtronic Inc.'s Quad Plus, Pisces Quad, Pisces Quad Compact, or 1×8 SubCompact, 1×8 Compact, and 1×8 Standard leads. Such percutaneous leads typically contain ring electrodes that apply an electrical stimulation signal to tissue in all directions around the ring. Accordingly, the amplitude of the signal (and thus the energy required from the signal generator) applied may be greater with percutaneous leads that surgical leads for occipital nerve therapies.

While not shown, the leads 400 depicted in FIGS. 14A-B may include engagement elements, as described above, and the distal arms 450, 451 may be implanted with the use of an engagement tool, as described above. For example, an incision may be made along the midline of the patient's neck, and the distal portions 450, 451 may be pushed in place subcutaneously using an engagement tool. The proximal portion of the lead 410 may be tunneled to a location in which an implantable signal generator is implanted or is to be implanted.

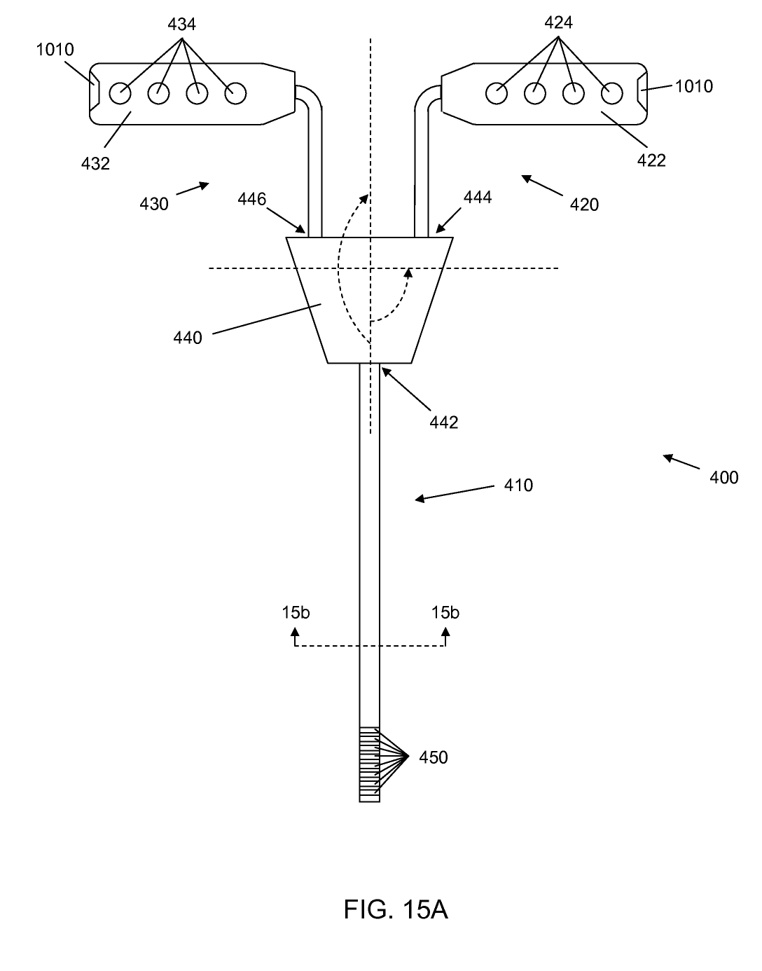

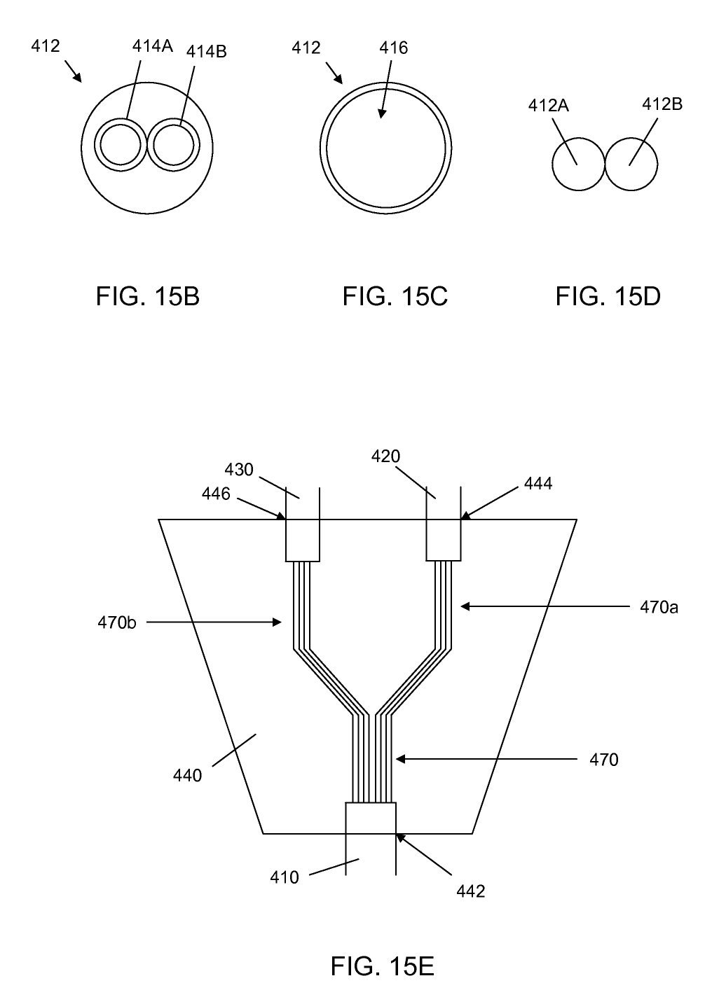

Various embodiments of bifurcated leads that may contain engagement features are described below with regard to FIGS. 15-19. Such leads may be used to apply electrical signal therapy to occipital nerves or other nerves or other targets. As with the non-bifurcated leads described above, it will be understood that the leads depicted in FIGS. 15-19 are merely examples of leads that may contain an engagement element.

Referring now to FIG. 15A, a side view of a representative bifurcated lead 400 is shown. The lead 400 includes a proximal portion 410 that includes a plurality of contacts 450 for electrically coupling to an electrical signal generator or a lead extension or an adaptor. The lead also includes first 420 and second 430 distal arms that contain an engagement element 1010 and electrodes 424, 434. The electrodes 424, 434 are electrically coupled to contacts 450 via conductors that run within lead 400 from the contacts 450 to the electrodes 424, 434. The lead 400 further includes a branch region 440 where the lead 400 transitions from the proximal portion 410 to the distal arms 420, 430. The branch region 440 may be of any suitable size and shape. In various embodiments, the branch region 440 has a volume of less than about 10 cubic centimeters; e.g., less than about 5 cubic centimeters.

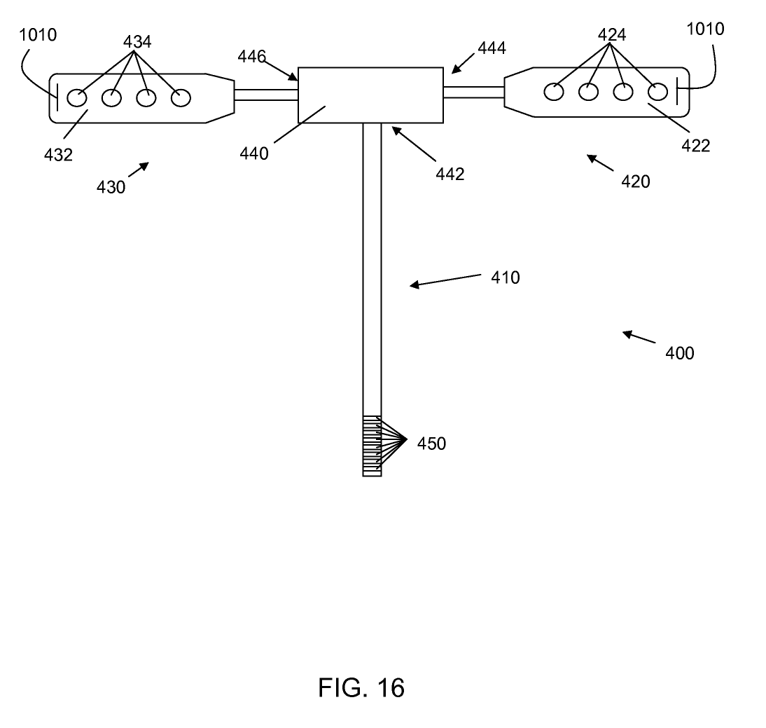

The branch region 440 includes a first entry region 442 where the proximal portion 410 of the lead enters the branch region. The branch region 440 also includes second 344 and third 346 entry regions where the first 420 and second 430 distal arms enter the branch region. A plane runs through the centers of the entry regions 442, 444, 446. The angle of either of the second 444 and third 446 entry regions from a line extending in the plane and aligned with the geometric center first entry point 442 as it extends to proximal portion 410 of the lead 400 is between about 90 degrees and 180 degrees. In some embodiments, the center of the second 444 or third 446 entry region is substantially perpendicular to the line extending in the plane and aligned with the geometric center first entry point 442 (see, e.g., FIG. 16). In some embodiments, the angle of the second 444 or third 446 entry region relative to the first entry point 442 is between about 110 degrees and about 160 degrees.

Referring now to FIG. 15B-D, which is a cross section of the proximal portion 410 of the lead 400 depicted in FIG. 15A taken along line 15b-15b, showing representative configurations. As shown in FIG. 15B, the proximal portion of the lead includes a lead body 412. The lead body 412 may include two lumens or tubes 414A, 414B (or any number of tubes or lumens, e.g. one for each conductor) through which or around which conductors (not shown) may run to connect proximal contacts with electrodes of the first and second distal arms. Of course, the lumens or tubes 414A, 414B may be solid and the conductors can run in separate tracks along the length of the proximal portion of the lead until connecting with the distal arms. Alternatively, as shown in FIG. 15C, the lead body 412 in the proximal portion may include a single lumen 416 or solid core (not shown) and the conductors (not shown) may run in a single track along the along the length of the proximal portion of the lead. Alternatively as shown in FIG. 15D, the proximal portion of the lead may include two attached lead bodies 412A, 412B through which separate channels of conductors (not shown) run. Of course, the lead body of the proximal portion of lead body may be configured in any other suitable manner.

Referring now to FIG. 15E, a representative example of a branch region 440 is shown in which the branch region 440 is transparent for purposes of illustration. In the depicted embodiment, a set of conductors 470 exit a lead body from the proximal portion 410 of the lead. The set of conductors 470 are separated into subsets 470a, 470b that independently enter lead bodies of the first 420 and second 430 distal arms. Any suitable manner of forming branch region 440 and separating conductors 470 for entry of subsets 470a, 470b into distal arms 420, 430 may be employed. For example, a lead body containing conductors 470 in proximal portion 410 may be formed. Additional lead bodies containing conductor subsets 470a, 470b forming distal arms 420, 430 may be formed. The conductor subsets 470a, 470b may be appropriately electrically coupled to the set of conductors 470 and branch region 440 may be overmolded over conductors 470, 470a, 470b, resulting in branch region 440 as depicted. Of course, any other suitable process may be employed to form branch region 440 and appropriately electrically couple proximal portion 410 of the lead to the distal arms 420, 430.

Referring now to FIG. 16, a side view of a representative lead 400 is shown. The lead 400 includes a proximal portion 410 including contacts 450, a first distal arm 420 having a region 422 containing an engagement element 1010 and electrodes 424, a second distal arm 430 having a region 432 containing an engagement element 1010 and electrodes 434, and a branch point 440 where the lead 400 transitions from the proximal portion 410 to the first 420 and second 430 distal arms. The distal arms 420, 430 exit the branch point 440 substantially perpendicular to the entry of the proximal portion 410 in the depicted embodiment. The distal portions 422, 432 containing the electrodes 424, 434 are paddle-shaped in the embodiment depicted in FIG. 16. Of course, distal portions containing the electrodes may have any suitable shape, such as cylindrical.

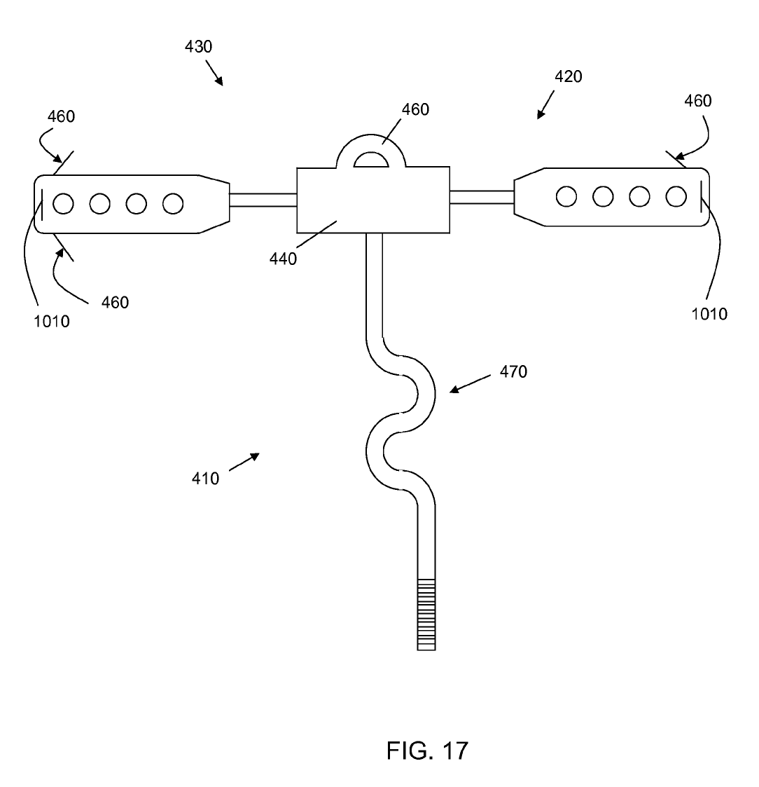

Referring now to FIG. 17, a lead 410 may include one or more anchors 460 for facilitating retention of the lead to tissue into which it is implanted. In FIG. 17, the anchors 460 are depicted as suture holes or tines, but the anchors may take any suitable form. In various embodiments, an anchor 460 is attached to branch region 440. As used herein, “attached” includes “integrally formed with.” For application of therapies to an occipital nerve, where proximal portion 410 is tunneled through the neck region of a subject, it may be desirable to securely anchor branch region 440 to tissue of the subject to prevent movement of the lead (and thus proximal portion 410) from causing movement of distal arms 420, 430 or portions thereof. In addition, it may be desirable for proximal portion to contain a strain relief feature to allow for stretching and movement of the next (and thus proximal portion 410) from transferring excessive force to branch region 440. For example, proximal portion 410 may include a sigma shaped portion 470, may be looped (not shown), or may be extensible. One or more anchors 460 may be attached to first 420 or second 430 distal arms or to portions thereof, such as the distal portions containing electrodes as depicted.

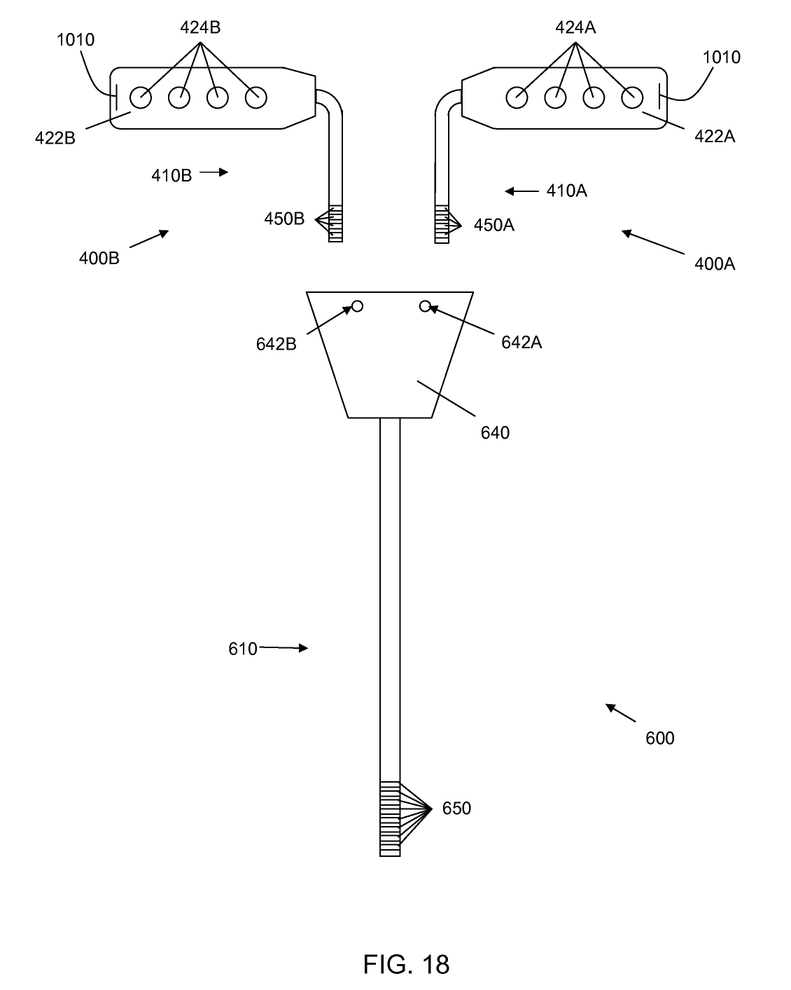

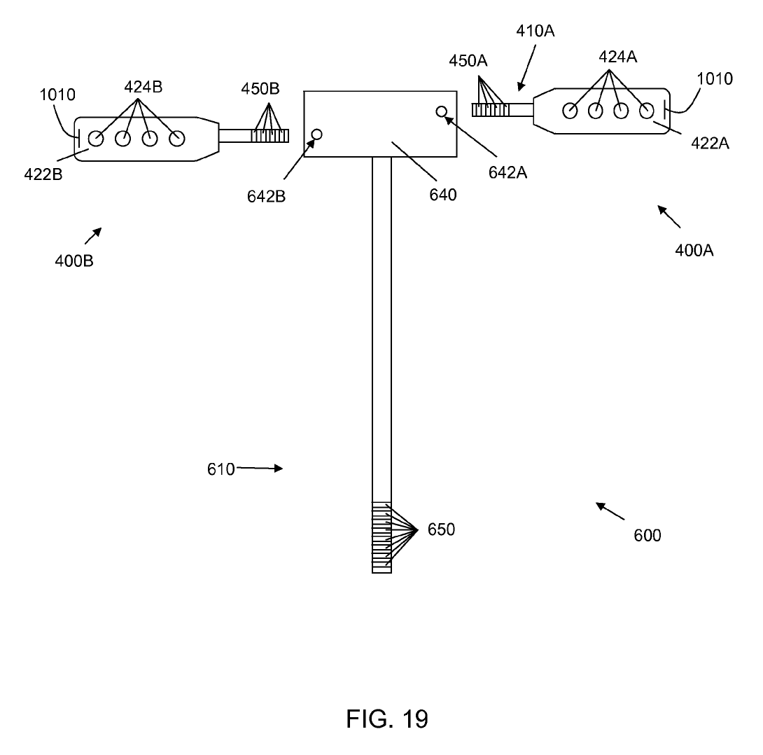

Referring now to FIGS. 18-19, a schematic drawing of bifurcating lead extensions 600 and associated leads 400A and 400B are shown. Bifurcating lead extensions 600 as described herein have many of the advantages discussed above with regard to bifurcating leads. For example, only one tunneling procedure is needed to proximal portion 610 of extension 600 to the site of implantation of signal generator. The proximal portion 610 of the extension 600 includes contacts 650 for electrical coupling the extension 600 to the signal generator. The distal portion of extension 600 includes a connector 640 containing two lead receptacles (not shown) having internal contacts for coupling to contacts 450A, 450B of leads 400A, 400B. The connector 600 may be of any suitable size and shape. In various embodiments, the connector 600 has a volume of less than about 10 cubic centimeters; e.g. less than about 5 cubic centimeters. Set screws 642A, 642B may be used to secure leads 400A, 400B in receptacles. Of course, any other suitable mechanism for securing leads 400A, 400B in receptacles may be employed. In the embodiment depicted in FIG. 19, the lead receptacles (not shown) are generally perpendicular to the angle of entry of the proximal portion 610 into connector 640.

Leads 400A, 400B include proximal portions 410A, 410B containing contacts 450A, 450B and distal portions 422A, 422B containing electrodes 424A, 424B and an engagement element 1010. By employing a bifurcating extension 600 and separate leads 400A, 400B standard introducer tools, such as needle introducers with lumens (provided that the distal portion of the lead fits within the lumen), may be used to position distal portion 424A, 424B of leads 400A, 400B. For bifurcating leads alternative methods for introducing distal portions may be desired.

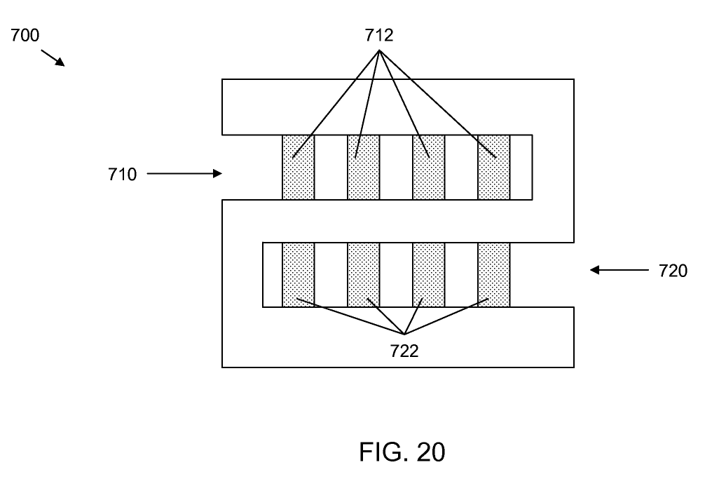

Referring now to FIG. 20, a schematic cross-section of a connector portion 700 of a lead extension; e.g. connector 640 as depicted in FIG. 19, is shown. Connector 700 includes first 710 and second 720 lead receptacles. The receptacles 710, 720 include openings on opposing ends of connector 700 for inserting leads in to the receptacles 710, 720 and include internal contacts 712, 722 for electrically coupling to contacts of leads when inserted into the receptacles 710, 720.

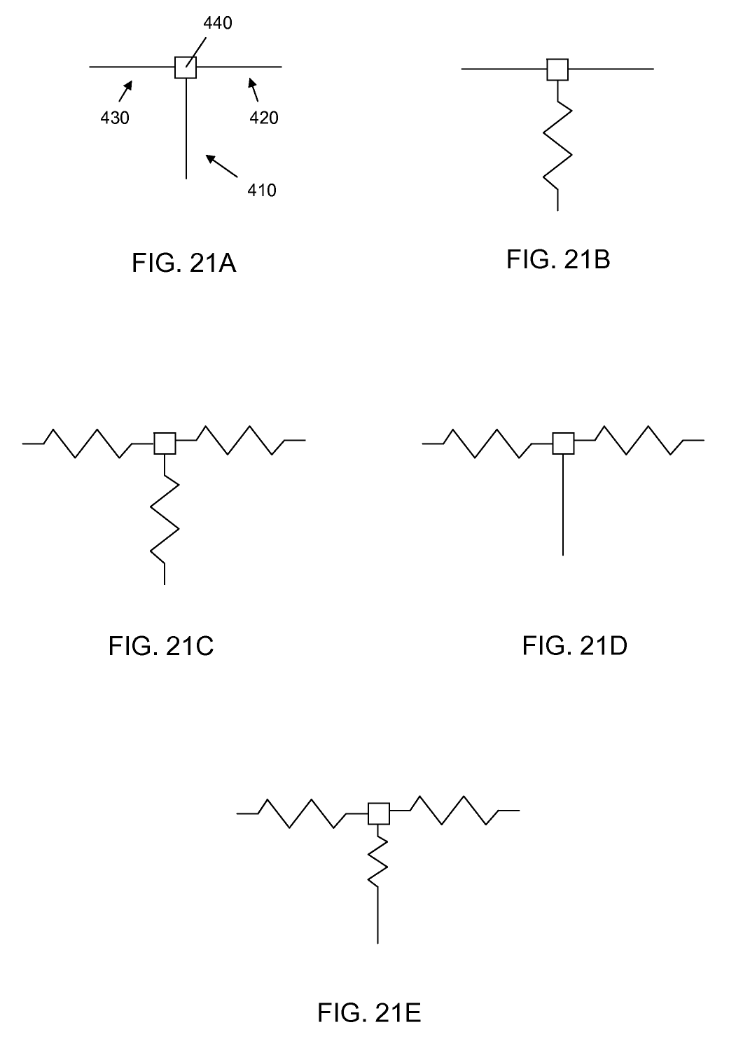

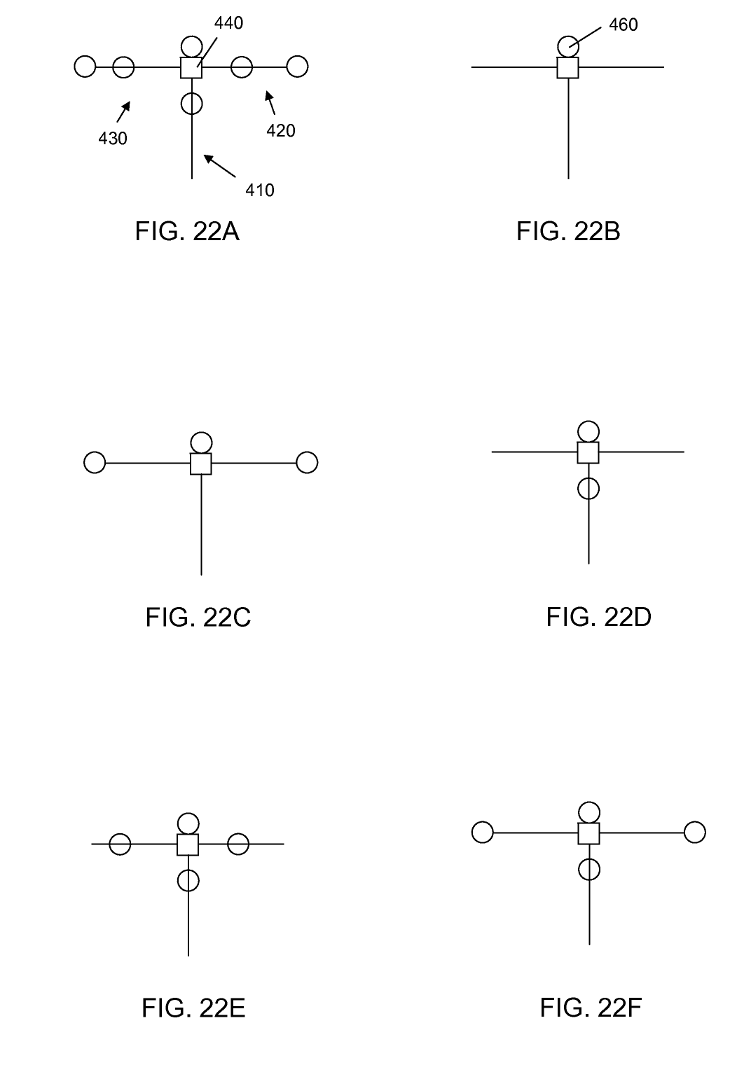

Referring now to FIGS. 21-22, various representative configurations of bifurcated leads are shown. However, it will be understood that the configurations presented may also be applied to bifurcating extensions. Further, while T-shaped configurations are depicted, it will be understood that such configurations are readily applicable to Y- or other shaped configurations. Engagement elements are not shown in the leads depicted in FIGS. 21-22, however, it will be understood that the leads may include engagements elements, e.g. as described above. In the embodiments depicted in FIGS. 21A-E, the bifurcated leads include a proximal portion 410 containing contacts (not shown), a branch region 440 and first 420 and second 430 distal arms containing electrodes (not shown). The squiggly lines depicted in FIGS. 21B-E represent extensibility of the lead of that squiggly portion. Extensibility may include a sigma shaped section, loops, or may otherwise be configured to be extensible. As depicted, proximal portion 410 or distal arms 420, 430 or portions thereof may be extensible.

As shown in FIGS. 22A-F, in which circles represent attached anchors 460, a bifurcated lead may include one or more attached anchor at nearly any location of the lead, such as the distal portion or along the length of a distal arm 420, 430, at a branch region 440, or anywhere along the proximal portion 410. It will be understood that possible combinations of the configurations shown in FIGS. 21-22 are contemplated, as are combinations of other figured depicted and discussed herein.

Thus, embodiments of LEAD WITH DISTAL ENGAGEMENT ELEMENT TO FACILITATE LEAD PLACEMENT are disclosed. One skilled in the art will appreciate that the leads, extensions, connectors, devices such as signal generators, systems and methods described herein can be practiced with embodiments other than those disclosed. The disclosed embodiments are presented for purposes of illustration and not limitation.

1. A method for pushing a distal portion of a lead through tissue of a patient, the distal portion of the lead including an electrode and an engagement element, the method comprising:
engaging the engagement element of the lead with an engagement tool;
advancing the tool distally relative to the lead to push the distal portion of the lead through the tissue.

2. A method according to claim 1, wherein the lead is pushed by distal advancement of the tool until the electrode is positioned in a desired location of the tissue.

3. A method according to claim 1, wherein the lead is in contact with the tissue as the lead is pushed through the tissue by the tool.

4. A system for implanting a lead, comprising:
a lead having a distal portion including an electrode and an engagement element; and
an engagement tool having a lead engagement feature and an elongate member extending from the lead engagement feature such that distal advancement of the elongate member when the lead engagement feature is engaged with the engagement element of the lead pushes the lead distally.

5. A system according to claim 4, wherein the engagement element comprises a cavity formed in the distal portion of the lead.

6. A system according to claim 4, wherein the distal portion of the lead comprises a paddle-shaped portion, wherein the engagement element is integrally formed with the paddle-shaped portion.

7. A system according to claim 4, wherein the elongate member of the engagement tool is curved to such that pulling on portion of the elongate member distal to the engagement feature causes a portion of the elongate member in proximity to the engagement feature to push the engagement feature.

8. An implantable medical lead comprising:
a proximal portion including a contact; and
a distal portion including a paddle-shaped portion, an electrode, and an engagement element configured to cooperate with a lead advancement tool to facilitate placement of the lead such that distal advancement of the tool relative to the lead pushes the lead distally,
wherein the electrode is electrically coupled to the contact, and
wherein the engagement element is integrally formed with the paddle-shaped portion.

9. A lead according to claim 8, wherein the distal portion further comprises one or more additional electrodes, and wherein the engagement member is distal to each of the electrodes.

10. A lead according to claim 8, wherein engagement member forms a cavity configured to receive a portion of the advancement tool.

11. A lead according to claim 8, wherein the lead is bifurcated and further comprises first and second distal arms, wherein the first distal arm comprises the distal portion.

12. A lead according to claim 11, wherein the second distal arm comprises a paddle-shaped portion including an engagement element.

13. A system comprising:
a lead comprising:
a proximal portion including a contact; and
a distal portion including a paddle-shaped portion, an electrode, and an engagement element configured to cooperate with a lead advancement tool to facilitate placement of the lead such that distal advancement of the tool relative to the lead pushes the lead distally,
wherein the electrode is electrically coupled to the contact, and
wherein the engagement element is integrally formed with the paddle-shaped portion; and
an electrical signal generator having a lead receptacle configured to receive the proximal portion of the lead and electrically couple to the contact of the lead.

14. A method for applying electrical signals to left and right occipital nerves of a patient, comprising:
(i) implanting a lead including a proximal portion, a first distal arm, a second distal arm, and a branch region between the proximal portion and the first and second distal arms, the proximal portion including first and second contacts, the first distal arm including an electrode electrically coupled to the first contact and a having an engagement element distal to the electrode, and the second distal arm including an electrode electrically coupled to the second contact and having an engagement element distal to the electrode, wherein implanting the lead comprises:
engaging the engagement element of the first distal arm with a first engagement tool and advancing the tool distally relative to the lead to push the distal arm of the lead through tissue of the patient until the electrode is positioned adjacent to the left occipital nerve, engaging the engagement element of the second distal arm with a second engagement tool and advancing the tool distally relative to the lead to push the distal arm of the lead through tissue of the patient until the electrode is positioned adjacent to the right occipital nerve, wherein the first and second engagement tools are the same or different;
(ii) operably coupling the first and second contacts of the proximal portion of the lead with an implantable signal generator; and
(iii) delivering a first signal generated by the signal generator to the left occipital nerve via the electrode of the first distal arm of the lead, and delivering a second signal generated by the signal generator to the right occipital nerve via the electrode of the second distal arm of the lead, wherein the first and second signal are the same or different.

15. A method according to claim 14, further comprising implanting the signal generator in the patient and tunneling the proximal portion of the lead through the patient to the site of the implanted signal generator.

16. A method according to claim 15, wherein further comprising making an incision in the back of the patient's neck, wherein the proximal portion of the lead is tunneled from the site of the incision to the site of the implanted signal generator.

17. The method according to claim 16, wherein the first distal arm is pushed from the site of the incision to through the tissue of the patient until the electrode is positioned such that it is capable of delivering a therapeutic electrical signal to the left occipital nerve, and wherein the second distal arm is pushed from the site of the incision to through the tissue of the patient until the electrode is positioned such that it is capable of delivering a therapeutic electrical signal to the right occipital nerve.
